(19) United States
(12) Patent Application Publication
(10) Pub. No.: US 2019/0314458 A1
(43) Pub. Date: Oct. 17, 2019

(54) METHODS OF TREATING HORMONAL DISORDERS ASSOCIATED WITH ANDROGEN DEFICIENCY USING A REPEAT MICROFLARE DOSING REGIMEN

(71) Applicant: Pieter Muntendam, Boxford, MA (US)

(72) Inventor: Pieter Muntendam, Boxford, MA (US)

(21) Appl. No.: 16/379,434

(22) Filed: Apr. 9, 2019

Related U.S. Application Data
(60) Provisional application No. 62/657,503, filed on Apr. 13, 2018.

(51) Int. Cl. A61K 38/22; A61K 31/4178

(52) U.S. Cl. CPC A61K 38/22; A61K 31/4178

(57) ABSTRACT

A method of treating a hormonal disorder associated with androgen deficiency is disclosed. The method includes administering a therapeutically effective dose of a composition having a GnRH agonist to a subject in need thereof. The therapeutically effective dose is sufficient to support a first flare of hormones. The method further includes allowing a time interval after the administration of the dose. The time interval is sufficient to support pituitary-gonadal axis recovery. The method further includes administering at least one subsequent dose of the composition to the subject after the time interval. The subsequent dose is sufficient to support a microflare of hormones that is less than, or equal to, a magnitude of the first microflare. Subsequent doses spaced by the time interval resulting in a repeat microflare sufficient to chronically elevate hormone levels and provide relief of signs or symptoms of androgen deficiency.

### RELATED APPLICATION

This application claims the benefit of U.S. Provisional Application No. 62/657,503, filed on Apr. 13, 2018. The entire teachings of the above application is incorporated herein by reference.

### TECHNICAL FIELD

The present disclosure relates to methods and compositions for the treatment of hormonal disorders associated with androgen deficiency.

### BACKGROUND

Male hypogonadism is a clinical disorder that results from failure of testis to produce normal levels of testosterone by disruption of one or more levels of hypothalamic-pituitary-gonadal (HPG) axis. Hypogonadotropic hypogonadism (HH) is a clinical condition that result from inadequate stimulation of testicular testosterone production. Androgen deficiency in aging male (ADAM) is the leading cause of hypogonadism as testosterone levels decline progressively after age of 40. In aging men, predominant form of testosterone deficiency (TD) is mixed with primary and secondary hypogonadism components. Further, HH may result from either absent or inadequate hypothalamic gonadotropin-releasing hormone (GnRH) secretion or failure of physiologic pituitary gonadotropin secretion, including follicle-stimulating hormone (FSH) and luteinizing hormone (LH). HH is most common form of hypogonadism in adult men. HH may result due to various congenital or acquired causes and can be differentiated accordingly. Congenital HH includes Kallmann syndrome, adrenal hypoplasia congenita, and idiopathic hypogonadotropic hypogonadism (IHH). Acquired HH can be structural, for example, caused by tumors and infiltrative disorders, or functional, for example, resulting from comorbidities, excessive exercise, dieting, or narcotic abuse among others. Common symptoms of HH can include decreased sexual libido, impaired erectile function, muscle weakness, increased adiposity, mood alterations, and fatigue.

Currently, the leading treatment of TD involves hormonal replacement therapy with testosterone (TRT). Oral delivery of testosterone is not possible due to rapid first pass metabolism and short half-life. Parenteral administration may involve injection therapy or transdermal in the form of a topical preparation or a patch. Use of the TRT is associated with an increased risk for cardiovascular complications including heart attack and stroke. Due to the risk of such serious complications, testosterone products are not approved for the treatment of low testosterone levels due to aging. Testosterone is FDA-approved as replacement therapy only for men who have low testosterone levels due to confirmed disorders of the testicles, pituitary gland, or brain. Examples of the disorders include failure of the testicles to produce testosterone because of genetic problems, or damage from chemotherapy or infection. In addition to the cardiovascular adverse events associated with TRT, TRT suppresses the HPG through a negative feed-back loop resulting in reduced endogenous production of testosterone resulting a dependency on the TRT. Despite these shortcomings, TRT is being used extensively in attempts to relieve symptoms of ADAM. The widespread use of TRT despite the well-known risks and complications associated with it is indicative of the unmet need. There is no approved method of treatment for TD that depends upon enhancing endogenous androgen production.

### SUMMARY

The present disclosure is directed to a method of treating a hormone-related condition associated with androgen deficiency in patients with hypogonadotropic hypogonadism (HH) or certain cases of androgen deficiency of the aging male (ADAM). The method uses stimulation of endogenous androgen production. Symptoms of androgenic deficiency disorders may include fatigue, depression, decreased sexual libido, sexual dysfunction, erectile dysfunction, hypogonadism, and alterations in mood and cognition. In a suitable candidate a first dose of a gonadotropin-releasing hormone (GnRH) agonist administered to the patient resulting in a testosterone burst or flare. The initial dose is followed by a time interval of sufficient duration, during which pituitary-gonadal axis sufficiently recovers to support a repeat flare of similar or reduced magnitude following a subsequent dose. Such treatment method of hormone-related condition associated with the androgen deficiency is referred as a “repeat microflare” (RmF) treatment method. Successful RmF requires a specific dose and interval range for each agonist (treatment parameters) and will result in chronic increase of testosterone levels over the pre-treatment level sufficient to result in relief of signs or symptoms of androgen deficiency. Outside the specific ranges for interval and dose, agonist therapy may cause downregulation or result in incomplete or unsatisfactory response.

In one aspect of the present disclosure, a method of treating a hormonal disorder associated with the androgen deficiency is provided. The method includes administering a therapeutically effective dose of a composition having the GnRH agonist to a subject in need thereof. The therapeutically effective dose is sufficient to support a first flare of hormones from the pituitary-gonadal axis. The method further includes allowing the time interval after the administration of the dose. The time interval is sufficient to support the pituitary-gonadal axis recovery. The method further includes administering a subsequent dose of the composition to the subject after the time interval. The subsequent dose is sufficient to support a microflare of hormones that is less than, or equal to, a magnitude of the first flare and which reoccurs with each subsequent dose.

In various embodiments of the present disclosure, the GnRH agonist includes at least one of buserelin, deslorelin, fertirelin, gonadorelin, goserelin, histrelin, leuprorelin or leuprolide, nafarelin, triptorelin, GnRH derivatives, optical isomers, pharmaceutically acceptable salts, formulations, hydrates, and any combination thereof. The hormones include testosterone, androstenedione, and any such intermediates and derivatives thereof. In the illustrated embodiment, the hormone is testosterone. The GnRH agonist is administered to the subject via at least one of, subcutaneous, oral, topical, rectal, intramuscular, transdermal, intranasal, inhalation and buccal administration. The GnRH agonist may also be administered with an absorption enhancer. Subsequent doses spaced by the time interval resulting in a repeat microflare sufficient to chronically elevate hormone levels and provide relief of signs or symptoms of androgen deficiency. The time interval and the subsequent dose may be individualized based on a test treatment involving one or more GnRH administrations. Further, the time interval and the subsequent dose may be individualized based on at least one of, hormonal assessment, symptoms, laboratory testing and physical assessment. The subsequent dose and the time interval may be periodically adjusted based on treatment response. The subsequent dose and the time interval are adjusted to maintain hormone levels in the subject within a desired range.

### BRIEF DESCRIPTION OF THE DRAWINGS

FIG. 1 illustrates a HPG axis and mechanism of action of hormones in a feedback loop; and

FIG. 2 is a graphical representation of repeat microflare treatment methods showing changes in testosterone levels over a period of preset time intervals in comparison to conventional GnRH agonist therapy resulting in gonadal suppression.

### DETAILED DESCRIPTION

The present disclosure demonstrated in this application is a mere illustration of the disclosure and various embodiments and forms may be prepared. Examples, therefore, given are only for illustration purposes without any intention to restrict the embodiments to a given set of examples. Specific structures, figures and functional aspects are provided merely to enable a person skilled in the art to perform the disclosure and should not be construed as limitations of the disclosure. The terms “include”, “includes”, “including”, “have”, “has,” “having”, “comprise”, “comprises”, “comprising” or the like should be generally understood as open-ended and non-limiting unless specifically stated otherwise. It should also be understood that the order of steps or order for performing certain actions can be changed so long as the intended result is obtained. Moreover, two or more steps or actions may be conducted simultaneously.

The present disclosure illustrates methods and compositions for treatment and prevention of a hormone-related condition associated with androgen deficiency in patients. The method includes administering a therapeutically effective dose of a composition having a gonadotropin-releasing hormone (GnRH) agonist to a subject in need thereof to treat hormone deficiency. The dose supports the microflare of hormones from a pituitary-gonadal axis of the subject. Further, the method includes administering at least one subsequent dose of the GnRH agonist composition followed by a time interval of sufficient duration, during which the pituitary-gonadal axis sufficiently recovers, to support a repeat microflare of similar or reduced magnitude of hormones. The term “subject” refers to a mammal such as a human. The term “subject” also includes a patient, and does not exclude an individual that is normal in all respects.

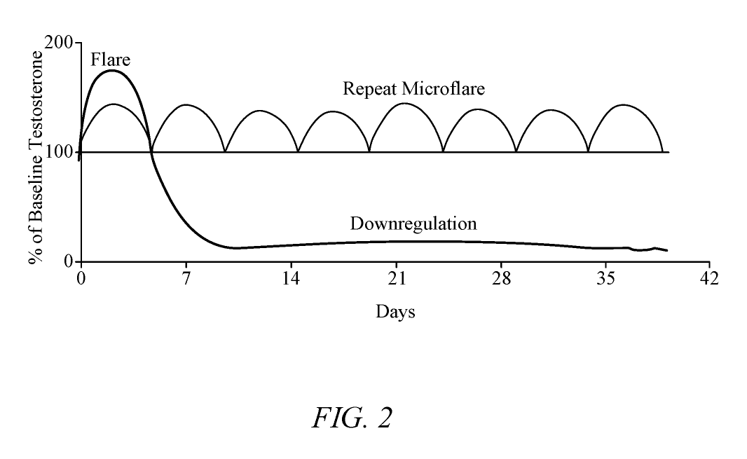

The term “microflare”, as shown in FIG. 2, refers to the method of repeatedly stimulating the pituitary-gonadal axis for release of hormones after a dosage regimen of the GnRH composition is administered to the subject. Administration of the GnRH agonist composition is followed by the time interval allowing sufficient time for the recovery of the pituitary-gonadal axis for the subsequent dose of the composition.

The term “flare”, as shown in FIG. 2, refers to an initial surge in hormonal level after administering a dose of the composition having the GnRH agonist, which may result in subsequent down-regulations of the pituitary-gonadal axis secretions over a period of time with repeat or continuous administration.

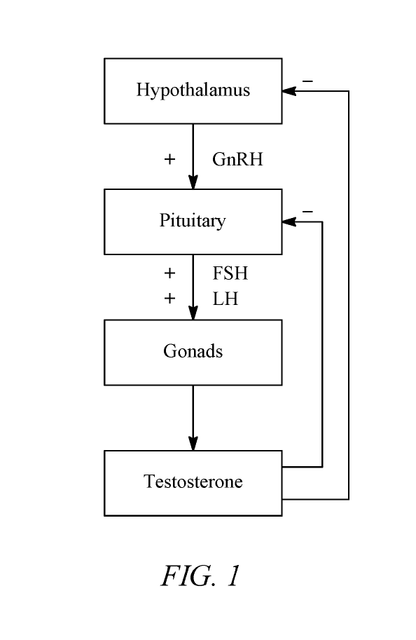

As used herein, “pituitary-gonadal axis” refers to a combination of pituitary gland and gonadal glands as a single endocrine unit or axis. The axis regulates reproduction, development as well as aging in humans. In normal condition, hypothalamus releases the GnRH that stimulates the pituitary gland to produce luteinizing hormone (LH) and follicle-stimulating hormone (FSH), which in turn stimulates the gonads to release estrogen and testosterone. As illustrated in the FIG. 1, the axis includes three parts, namely, hypothalamus, pituitary and gonads and is referred to as “hypothalamus-pituitary-gonadal axis” or “HPG axis”. It is understood that any reference to the “hypothalamus-pituitary-gonadal axis” or the “HPG axis” may include any part of the axis, and any reference to the axis may include all the parts or any two parts of the axis formed by hypothalamus, pituitary gland, and gonadal glands.

As used herein, “individualization” refers to defining the time interval and drug dosage regimens based on pharmacokinetics and pharmacodynamics analyses. Individualization may be based on test treatment, hormonal assessment, symptoms, laboratory testing, physical assessment, biochemical assessment or any such method that provides desired results in the subject for safe and effective management of the diseases or disorders. Individualization may include periodic changes and adjustments as may be needed in the subsequent doses and the time intervals based on treatment response. It is understood that any modification in the dose and the time interval may include all types of adjustments or repeat of the initial dose and time interval based on the treatment response. The term “periodically” refers to adjustments and modifications in time interval after an initial dose of the composition having the GnRH agonist is administered to the subject based on treatment response.

As used herein, “hormonal disorder” refers to any diseases, disorders, syndromes, or symptoms that may result due to gonadotropic deficiency including, but not limited to, androgen deficiency, hypoandrogenism, hypogonadotropic hypogonadism (HH), Kallmann syndrome, idiopathic hypogonadotropic hypogonadism (IHH), androgen deficiency in the aging male (ADAM), or any such disorders involving secretions from all or any part of the HPG axis. The term “symptoms” refers to decrease in sexual libido, sexual dysfunction, erectile dysfunction, depression, fatigue, hypogonadism, alteration in mode and cognition, and other indications that are associated with conditions of hormonal deficiency.

As used herein, the term “sufficient” in relation with the dose refers to minimum amount of the therapeutically effective dose of the GnRH agonist that results in the release of hormones from the gonads. Also, as used herein, the term “sufficient” in relation with the time interval refers to the time duration during which the pituitary-gonadal axis recovers for the subsequent dose after an initial or repeat dose of the composition having the GnRH agonist is administered to the subject.

As used herein, “preventing or treating” refers to partially or completely alleviating and/or ameliorating the condition and/or symptoms thereof, and/or preventing diseases or symptom re-occurrence or halting its progression. The present disclosure, accordingly, includes a method of providing, to a patient, a combination product that includes a compound or therapeutic composition of the present disclosure in combination or association with a pharmaceutically acceptable carrier. The term “pharmaceutically acceptable” refers to a substance that is acceptable for use in pharmaceutical applications from a toxicological perspective and does not adversely interact with the active ingredient. Accordingly, pharmaceutically acceptable carriers are compatible with the other ingredients in the formulation and are biologically acceptable. Supplementary active ingredients can also be incorporated into the pharmaceutical compositions.

Treatment methods, compounds and therapeutic compositions of the present disclosure can be useful for preventing or treating pathological condition or disorders in a patient. For example, compounds and therapeutic compositions of the present disclosure can be administered alone or in combination with other therapeutically effective compounds or therapies for the treatment of a pathological condition or disorder. The term “therapeutically effective” refers to a substance or an amount that elicits a desirable biological activity or effect through administrating the composition having the GnRH agonist and reducing or arresting hormonal diseases and disorders or symptoms.

As used herein, “administration” refers to oral, or parenteral including intravenous, subcutaneous, topical, transdermal, intradermal, transmucosal, intraperitoneal, intracapsular, intraorbital, intracardiac, transtracheal, subcutaneous, subcuticular, intraarticular, subcapsular, subarachnoid, intraspinal, epidural and intrasternal, injection and infusion, rectal, intramuscular, transdermal, intranasal, inhalation and buccal administration. When administered for the treatment of a specific hormone related diseases or disorders, it is understood that an effective dosage can vary depending upon factors such as the particular compound or therapeutic composition utilized, the mode of administration, severity of the condition being treated, and the various physical factors related to the patient being treated. In therapeutic applications, a compound or therapeutic composition of the present disclosure can be provided to a patient already suffering from a disease, for example, HH. The composition or therapeutic composition is administered to the patient in an amount sufficient to at least partially ameliorate the symptoms of the disorders, diseases or syndrome and its complications and halt or slow down the disease progression. If administered to a human suffering from the condition prior to clinical manifestation, the administration of the therapeutic composition may prevent the first clinical manifestation or delay its onset.

As used herein, “compound” refers to the compound itself and its pharmaceutically acceptable salts, derivatives, optical isomers, hydrates and esters, unless otherwise understood from the context of the description or expressly limited to one particular form of the compound, i.e., the compound itself, or a pharmaceutically acceptable salt, derivatives, optical isomers, hydrate or ester thereof.

The present disclosure provides methods of administration of the composition including the GnRH agonist or one or more of its analogues or variations or precursors to the subject with hormone related diseases or disorders. The hormone related diseases or disorders include, but are not limited to, androgen deficiency, hypoandrogenism, HH, hypogonadism, Kallmann syndrome, idiopathic hypogonadotropic hypogonadism (IHH), androgen deficiency in the aging male (ADAM), or any such disorders involving diminished secretions from the HPG axis. The method of treatment is provided for the hormonal disorders related to androgenic deficiency. The composition having the GnRH agonist is administered to the subject either suffering from hormonal deficiency or has such symptoms that indicate a hormonal disorder. The composition having the GnRH agonist administered to the subject results in the flare or burst of gonadal hormones. This is followed by the time interval that is sufficient to allow the pituitary-gonadal axis to recover prior to a subsequent dose of the composition having the GnRH agonist. The subject is administered at least one more dose of the composition to support the repeat microflare of hormones in which the magnitude of the microflare is less or equal to first flare. In most embodiments, multiple subsequent doses are administered.

In some embodiments, the magnitude of the microflare may be defined between 0.001 nanograms per deciliter (ng/dL) and 200 ng/dL of serum testosterone level based on the various test assessments. In some examples, the magnitude of the microflare may be 0.01 ng/dL to 10 ng/dL, 10 ng/dL to 50 ng/dL, or 50-200 ng/mL of serum testosterone level.

The time interval between the dosage regimens may be individualized in such a manner that the treatment with the composition having the GnRH agonist is therapeutically effective and safe for the subject. The individualization of the time interval is based on the treatment response of the subject after treating with the composition having the GnRH agonist at least once. In some embodiments, multiple doses are administered at equal or unequal time intervals. The subsequent doses and the time intervals may be periodically adjusted based on the treatment response.

The dose of the composition and the time interval may be individualized and modified based on the assessment of at least one of the hormonal assessment, laboratory testing, physical assessment, clinical and biochemical assessment, symptoms of the diseases or disorders which may include, but are not limited to, fatigue, depression, decreased sexual libido, sexual dysfunction, erectile dysfunction, hypogonadism, and alterations in mood and cognition.

The subsequent dose of the composition is administered to the subject such that the hormone levels in the body are maintained within a desired range. The time interval between the doses is adjusted to support the desired range in level of hormones in the subject. The term “desired range” refers to hormone levels in or close to the physiologic range as produced by the body upon administration of the GnRH agonist for treating the diseases or disorders. The desired range may be adjusted based on various assessments including hormonal assessment, symptoms, laboratory testing, physical assessment and patient response. The desired range may be 280 ng/dL to 1100 ng/dL of serum hormone level. In some examples, the desired range may be less than 280 ng/dL or more than 1100 ng/dL of serum hormone level based on the test assessment.

The treatment method of the present disclosure provides the composition including the GnRH agonist effecting a response from the pituitary-gonadal axis with the microflare of hormones including, but not limited to testosterone, androstenedione, and any intermediates and derivatives thereof. Also, the treatment method of the present disclosure provides the composition effecting a gonadal response with the microflare of the hormone testosterone. The treatment method of the present disclosure may alleviate the symptoms or reduce the risk of the subject from developing the hormonal disorder.

The GnRH agonist of the composition for the treatment of androgenic deficiency is at least one of, buserelin, deslorelin, fertirelin, gonadorelin, goserelin, histrelin, leuprorelin, nafarelin, triptorelin, GnRH agonist derivatives, optical isomers, pharmaceutically acceptable salts, formulations, hydrates, and any combination thereof. The GnRH agonist may be administered with an absorption enhancer or penetration accelerator. As used herein, “absorption enhancer” refers to an agent or agents known to accelerate absorption or penetration of the GnRH agonist or the composition having the GnRH agonist. Such an enhancer may be useful in accelerating the absorption via oral, subcutaneous, topical, rectal, intramuscular, transdermal, intranasal, inhalation and buccal route.

The symptoms of the disorders may be reduced by at least about 5% to about 99% as compared to an untreated subject. The compound may be administered to the subject in various forms including an ingestible composition, an injection, a transdermal patch, a suppository, solid foodstuff, powder form, juices, tablets, capsule, lozenge, gel caps, syrup, inhalation, emulsion, suspension, soft gel, creams, lotions, and aerosols. The composition may be administered via enteral or parental route including oral, sublingual, rectal, intravenous, subcutaneous, topical, transdermal, intradermal, transmucosal, intraperitoneal, intramuscular, intracapsular, intraorbital, intracardiac, transtracheal, subcutaneous, subcuticular, intraarticular, subcapsular, subarachnoid, intraspinal, epidural and intrasternal injection and infusion. In some embodiments of the present disclosure, the composition may be included in a foodstuff. The foodstuff may be at least one of a fruit or vegetable product, such as a baked good, a beverage, and a mixture of raw and/or cooked fruits and/or vegetables. The foodstuff may be fortified with the composition having the GnRH agonist. In some cases, the foodstuff may be fortified with an amount of the composition having the GnRH agonist that is sufficient to have a therapeutic effect on the subject.

In the present disclosure, the composition may include a plurality of one or more lower alcohols such as ethanol or isopropanol, a penetration enhancing agent, a thickener, and water. Additionally, the composition of the present disclosure may include salts, emollients, stabilizers, antimicrobials, and propellants. The typical dosages of the compounds and compositions of the present disclosure may vary within a wide range depending on factors, including but are not limited to, route of administration, treatment stage, body weight, age and general condition of the subject or the patient.

The pharmaceutical composition of the present disclosure may also contain adjuvants, diluents, excipients and/or carriers, known in the art, compatible with the compounds and other ingredients of the pharmaceutical composition, and not deleterious to the recipient thereof.

The method of treatment as disclosed, has several advantages. For example, as illustrated in FIG. 2, the method supports generating repeat microflares instead of a single flare. Moreover, the method prevents the occurrence of downregulation resulting in gonadal suppression in contrast to the desired stimulation. In addition, the method allows for sufficient recovery of the pituitary-gonadal axis during treatment.

It is understood that the examples, embodiments and teachings presented in this application are described merely for illustrative purposes. Any variations or modifications thereof are to be included within the scope of the present application as discussed.

What is claimed is:

1. A method of treating a hormonal disorder associated with androgen deficiency, the method comprising:
(a) administering to a subject in need thereof, a therapeutically effective dose of a composition having a gonadotropin-releasing hormone (GnRH) agonist, wherein the therapeutically effective dose is sufficient to support a first flare of hormones;
(b) allowing a time interval after the administration, wherein the time interval is sufficient to support pituitary-gonadal axis recovery; and
(c) administering to the subject, at least one subsequent dose of the composition after the time interval, wherein the subsequent dose is sufficient to support a microflare of hormones that is less than, or equal to, a magnitude of the first flare, the combination of flare and repeat microflares resulting in an improvement of symptoms of androgen deficiency.

2. The method of claim 1, wherein the disorder associated with the androgen deficiency comprises hypogonadotropic hypogonadism (HH).

3. The method of claim 2, wherein the hypogonadotropic hypogonadism is Kallmann syndrome.

4. The method of claim 1, wherein the disorder comprises androgen deficiency in aging male (ADAM).

5. The method of claim 4, wherein symptoms of the ADAM comprises fatigue, depression, decreased sexual libido, sexual dysfunction, erectile dysfunction, hypogonadism, and alterations in mood and cognition.

6. The method of claim 1, wherein the GnRH agonist comprises at least one of, buserelin, deslorelin, fertirelin, gonadorelin, goserelin, histrelin, leuprorelin, nafarelin, triptorelin, GnRH agonist derivatives, optical isomers, pharmaceutically acceptable salts, formulations, hydrates, and a combination thereof.

7. The method of claim 1 further comprising, adjusting the subsequent dose and the time interval to maintain hormone levels in the subject within a desired range.

8. The method of claim 1, wherein the hormones comprise testosterone, androstenedione and intermediates and derivatives thereof.

9. The method of claim 1, wherein the hormone is testosterone.

10. The method of claim 1 further comprising, individualizing the time interval and the subsequent dose based on a test treatment.

11. The method of claim 10 further comprising, individualizing the time interval and the subsequent dose based on at least one of, hormonal assessment, symptoms, laboratory testing and physical assessment.

12. The method of claim 10 further comprising, periodically adjusting the subsequent dose and the time interval based on treatment response.

13. The method of claim 1 further comprising, administering the GnRH agonist to the subject via at least one of, subcutaneous, oral, topical, rectal, intramuscular, transdermal, intranasal, inhalation and buccal administration.

14. The method of claim 1 further comprising, administering the GnRH agonist with an absorption enhancer.
